SEQUENCE LISTING (6 sequences)

SEQ ID NO: 1 - protein, 278 aa, Rattus rattus
MGSPVFRRPFCHLSTYSLLWAIAAVALSTAQVEVVTQDERKLLHTTASLRCSLKTTQEPLIVTWQKKKAVGPENMVTYSKAHGVVIQPTYKDRINITELGLLNTSITFWNTTLDDEGCYMCLFNMFGSGKVSGTACLTLYVQPIVHLHYNYFEDHLNITCSATARPAPAISWKGTGSGIENSTESHSHSNGTTSVTSILRVKDPKTQVGKEVICQVLYLGNVIDYKQSLDKGFWFSVPLLLSIVSLVILLVLISILLYWKRHRNQERGESSQGMQRMK

SEQ ID NO: 2 - protein, 278 aa, Mus musculus
MGSLVFRRPFCHLSTYSLIWGMAAVALSTAQVEVVTQDERKALHTTASLRCSLKTSQEPLIVTWQKKKAVSPENMVTYSKTHGVVIQPAYKDRINVTELGLWNSSITFWNTTLEDEGCYMCLFNTFGSQKVSGTACLTLYVQPIVHLHYNYFEDHLNITCSATARPAPAISWKGTGTGIENSTESHFHSNGTTSVTSILRVKDPKTQVGKEVICQVLYLGNVIDYKQSLDKGFWFSVPLLLSIVSLVILLILISILLYWKRHRNQERGESSQGMQRMK

SEQ ID NO: 3 - protein, 326 aa, Mus musculus
MFCFWRTSALAVLLIWGVFVAGSSCTDKNQTTQNNSSSPLTQVNTTVSVQIGTKALLCCFSIPLTKAVLITWIIKLRGLPSCTIAYKVDTKTNETSCLGRNITWASTPDHSPELQISAVTLQHEGTYTCETVTPEGNFEKNYDLQVLVPPEVTYFPEKNRSAVCEAMAGKPAAQISWSPDGDCVTTSESHSNGTVTVRSTCHWEQNNVSDVSCIVSHLTGNQSLSIELSRGGNQSLRPYIPYIIPSIIILIIIGCICLLKISGFRKCKLPKLEATSAIEEDEMQPYASYTEKSNPLYDTVTKVEAFPVSQGEVNGTDCLTLSAIGI

SEQ ID NO: 4 - protein, 249 aa, Mus musculus
MHALGRTPALTLLIFIYNFVSVYTIVSVQMGTKARLCCRSIPLTKAVLITWIIKPRGQPSCIMAYKVETKETNETCLGRNITWASTPDHIPDLQISAVALQHEGNYLCEITTPEGNFHKVYDLQVLVPPEVTYFLGENRTAVCEAMAGKPAAQISWTPDGDCVTKSESHSNGTVTVRSTCHWEQNNVSAVSCIVSHSTGNQSLSIELSRGTTSTTPSLLTILYVKMVLLGIILLKVGFAFFQKRNVTRT

SEQ ID NO: 5 - protein, 275 aa, Mus musculus
MHALGRTLALMLLIFITILVPESSCSVKGREEIPPDDSFPFSDDNIFPDGVGVTMEIEIITPVSVQIGIKAQLFCHPSPSKEATLRIWEITPRDWPSCRLPYRAELQQISKKICTERGTTRVPAHHQSSDLPIKSMALKHDGHYSCRIETTDGIFQERHSIQVPGENRTVVCEAIASKPAMQILWTPDEDCVTKSKSHNDTMIVRSKCHREKNNGHSVFCFISHLTDNWILSMEQNRGTTSILPSLLSILYVKLAVTVLIVGFAFFQKRNYFRWI

SEQ ID NO: 6 - protein, 270 aa, Mus musculus
MHALGRIPTLTLLIFINIFVSGSSCTDENQTIQNDSSSSLTQVNTTMSVQMDKKALLCCFSSPLINAVLITWIIKHRHLPSCTIAYNLDKKTNETSCLGRNITWASTPDHSPELQISAVALQHEGTYTCEIVTPEGNLEKVYDLQVLVPPEVTYFPGKNRTAVCEAMAGKPAAQISWTPDGDCVTKSESHSNGTVTVRSTCHWEQNNVSVVSCLVSHSTGNQSLSIELSQGTMTTPRSLLTILYVKMALLVIILLNVGFAFFQKRNFART

(19) United States
(12) Patent Application Publication
(10) Pub. No.: US 2010/0104582 A1
(43) Pub. Date: Apr. 29, 2010

(54) CD200 and its receptor, CD200R, modulate bone mass via the differentiation of osteoclasts

(75) Inventors: Agnes Vignery, Branford, CT (US); Juan Zhang Ke, Newbury Park, CA (US); Jun Li, Danbury, CT (US)

Correspondence Address: MICHAEL P. MORRIS;BOEHRINGER INGELHEIM USA CORPORATION, 900 RIDGEBURY ROAD, P O BOX 368, RIDGEFIELD, CT, 06877-0368, US

(73) Assignees: BOEHRINGER INGELHEIM INTERNATIONAL GMBH, Ingelheim (DE); YALE UNIVERSITY, New Haven, CT (US)

(21) Appl. No.: 12/521,363

(22) PCT Filed: Jan. 10, 2008
(86) PCT No.: PCT/US08/50708

§ 371 Date: Jul. 23, 2009

Related U.S. Application Data
(60) Provisional application No. 60/880,094, filed on Jan. 11, 2007.

(51) Int. Cl. A61K 39/395; C07K 14/00; C07K 16/00; A61K 38/14; C12Q 1/02; A61P 19/10; A61P 35/00; A61P 29/00

(52) U.S. Cl. 424/172.1; 530/395; 530/389.1; 514/8; 435/29

(57) ABSTRACT

Disclosed are methods and compositions relating to CD200 and its receptor, CD200R which modulate bone mass via the differentiation of osteoclasts.

### APPLICATION DATA

This application claims benefit to U.S. provisional application Ser. No. 60/880,094 filed Jan. 11, 2007.

This work was supported by funds from the NIH (grant no. DE12110 to A.V.).

### INTRODUCTION

Multinucleate osteoclasts originate from the fusion of mononuclear phagocytes and play a major role in the resorption of bone (Vignery, 2005a, b, c). Osteoclasts are essential for both the development and the remodeling of bone, and increases in the number and/or activity of osteoclasts lead to diseases that are associated with generalized bone loss, such as osteoporosis, and others that are associated with localized bone loss, such as rheumatoid arthritis and periodontal disease. Since fusion is a key step in the differentiation of osteoclasts, a detailed understanding of the molecular mechanism of macrophage fusion should help us to develop strategies to prevent bone loss.

The adhesion of cells to one another that precedes fusion appears to involve a set of proteins similar to those exploited by viruses for fusion with host cells (Hernandez et al, 1997). It has been postulated, moreover, that viruses usurped the fusion-protein machinery from their target cells (Vignery, 2000). It is now generally accepted that virus-cell fusion requires both an attachment mechanism and a fusion peptide. An example of such fusion involves gp120 of the human immunodeficiency virus (HIV), which binds to CD4 on T lymphocytes and macrophages (Dalgleish et al., 1984; Klatzmann et al., 1984), while the fusion molecule gp40, which is derived from the same precursor (gp160) as gp120, is thought to trigger the actual fusion event. We postulated previously (Saginario et al, 1995) that the fusion machinery employed by macrophages is similar to that used by viruses to infect cells. In 1998, we reported that the expression of MFR/SIRPα is induced transiently in macrophages at the onset of fusion (Saginario, 1995). MFR/SIRPα and its receptor, CD47, belong to the superfamily of immunoglobulins (IgSF), as does CD4, and their interaction plays a role in the recognition of self and in the fusion of macrophages (Han, 2000). To gain further insight into the mechanism of macrophage fusion, we subjected fusing alveolar macrophages from rats to genome-wide oligonucleotide microarray analysis, and we discovered the expression of CD200 de novo at the onset of fusion.

CD200 belongs to the IgSF and has a short cytoplasmic tail. It is expressed on various types of mouse and human cells (see Minas and Liversidge, 2006, for a review) and on mouse osteoblasts (Lee et al., 2006), but not on macrophages. By contrast, the receptor for CD200 (CD200R), which, resembling CD200, contains two IgSF domains, is expressed predominantly in myeloid cells and includes an intracellular domain that mediates downstream signaling. Hence, CD200-CD200R has a pattern of expression similar to that of MFR/SIRPα-CD47 in that CD200, like CD47, is widely expressed while CD200R, like MFR/SIRPα, is expressed predominantly in cells that belong to the myeloid lineage. Therefore, we postulated that the CD200-CD200R axis might play a role in the fusion of macrophages and that mice that lack CD200 would have a defect in macrophage fusion and, as a result, in both osteoclast differentiation and bone remodeling.

We found that the expression of CD200 was strongly induced in macrophages at the onset of fusion, and that osteoclasts deficient in CD200 had a defect in differentiation and in signaling downstream of RANK, which is essential for osteoclastogenesis. We also found that CD200-deficient mice had a higher bone density and a lower number of osteoclasts than wild-type mice. Together, our observations indicate that the CD200-CD200R axis plays a central role in the fusion of macrophages and the formation of osteoclasts.

### DESCRIPTION OF THE FIGURES

FIG. 1: Rat alveolar macrophages and mouse bone marrow-derived macrophages express CD200 upon multinucleation. Freshly isolated rat alveolar macrophages were plated at confluency over 50% of the surface of each well, to promote fusion and multinucleation. After five days, they were subjected to immunohistochemical analysis. Note that mononucleated macrophages were positive for MFR/SIRPα and CD44 but not for CD200 (bar=1 mm). Also note that multinucleate rat alveolar macrophages contained hundreds of nuclei that were stained with DAPI (blue). Freshly isolated rat alveolar macrophages were plated as in A and subjected to Western blotting analysis at the indicated times. Note that CD200 was not detected in macrophages for the first 24 h. Mouse bone marrow-derived macrophages were cultured in the presence of M-CSF (30 ng/ml) and RANKL (100 ng/ml) for the indicated times to induce the differentiation of multinucleate osteoclasts. Cells were analyzed by RT-PCR. Note that mouse bone marrow-derived macrophages expressed transcripts for CD200 receptor I (CD200RI) but not for CD200. The abundance of CD200 mRNA relative to that of GAPDH, in response to M-CSF (30 ng/ml) and increasing doses of RANKL was determined (bars represent standard deviations; n=3). Mouse bone marrow-derived macrophages were cultured in the presence of M-CSF (30 ng/ml) and RANKL (100 ng/ml) for the indicated times to induce the differentiation of multinucleate osteoclasts. Cells were subjected to Western blotting analysis using antibodies directed against the indicated antigens.

FIG. 2: Flow-cytometric analysis (in a fluorescent-activated cell sorter, FACS) of the expression of CD200. Mouse bone marrow-derived macrophages were isolated from CD200^{+/+} and CD200^{-/-} mice, cultured in the presence of M-CSF (30 ng/ml) and RANKL (100 ng/ml) and subjected to flow-cytometric analysis at the indicated times with an antibody directed against CD200 and a control isotype antibody. Bone marrow-derived macrophages expressed increasing amounts of CD200 with time in the presence of M-CSF and RANKL, which promote fusion, multinucleation and osteoclastogenesis.

FIG. 3: The absence of CD200 increases bone density. Two-month-old male and female CD200-deficient mice had a higher spinal bone-mineral density than wild-type mice (PIXImus/DEXA; n=8).

FIG. 4: pQCT analysis of distal femurs and femoral shafts from two-month-old CD200-deficient and wild-type mice. Note that the femoral shaft from both male and female CD200-deficient mice had increased total bone density, while only female CD200-deficent mice had decreased trabecular area. The distal femurs from both male and female CD200-deficient mice had increased total bone density. By contrast, the trabecular area and the periosteal circumference increased in CD200-deficient male and decreased in CD200-deficient female as compared to wild types.

FIG. 5: Toluidine blue-stained sections of proximal tibiae from two-month-old CD200-deficient male and female mice and wild-type mice (bar=1 mm). Histomorphometric analysis of proximal tibiae from two-month-old CD200-deficient male and female mice. Both male and female CD200-deficient mice had an increased bone volume (BV/TV), and decreased osteoclastic surface relative to bone surface (Oc.S/BS). Female CD200-deficient mice also had a decreased osteoblastic surface (Ob.S/BS). MicroCT analysis of distal femurs from six-month-old male and female CD200-deficient mice. Note the increased density of trabeculae inside the distal femur of CD200-deficient male and female mice as compared to wild types. The widest diameter of the bone sections correspond approximately about 3 mm.

FIG. 6: Osteoblasts do not express CD200R and neither osteoblasts nor pre-osteoclasts are affected by the absence of CD200. Bone marrow cells from six- to eight-week-old CD200-deficient and wild type mice were plated in 24-well plates (5×10⁶ cells/well) and cultured for 9 to 11 days in □-MEM supplemented with ascorbic acid (50 μg/ml) and β-glycerophosphate (10 mM) to acquire the osteoblast phenotype. Cell lysates were analyzed for alkaline phosphatase activity and protein concentration (SD; n=6). Osteoblasts were examined for alkaline phosphatase activity and stained for calcium with alizarin red S to allow quantitation of the number of nodules per well (SD; n=6). Cells were subjected to Western blotting analysis with antibodies directed against mouse CD200, CD200R and GAPDH. Bone marrow-derived macrophages from six-week-old CD200-deficient and wild-type mice were cultured in the presence of M-CSF (30 ng/ml) for two days prior to be subjected to flow-cytometric analysis with antibodies directed against c-fms, Mac-1 and C-kit, as surface markers. Note that the absence of CD200 did not affect the number of osteoclast precursor cells (left panel, bars=SD; n=5). Bone marrow-derived macrophages from six-week-old CD200-deficient mice were cultured in the presence of M-CSF (30 ng/ml) and increasing concentrations of RANKL for 5 days to induce the differentiation of osteoclasts. Bone marrow macrophages that lacked CD200 formed fewer osteoclasts than wild-type cells (right panel; bars=SD; n=5).

FIG. 7: In osteoclasts deficient in CD200, the activation of signaling molecules downstream of RANK is suppressed. Bone marrow macrophages isolated from CD200-deficient and wild-type mice were cultured in the presence of M-CSF (5 ng/ml) for 12-18 h. Non-adherent cells were further cultured for two days in 24-well dishes, starved for 2 h, and then stimulated with 50 ng/ml RANKL for the indicated times. Cells were lysed in Laemmli's sample buffer for SDS-PAGE analysis, supplemented with inhibitors of proteases and phosphatases' and subjected to Western blotting analysis with antibodies directed against the indicated antigens. The activation, by phosphorylation, of IkB and JNK was less extensive in cells that lacked CD200 than in wild-type cells. This experiment was repeated three times with similar results.

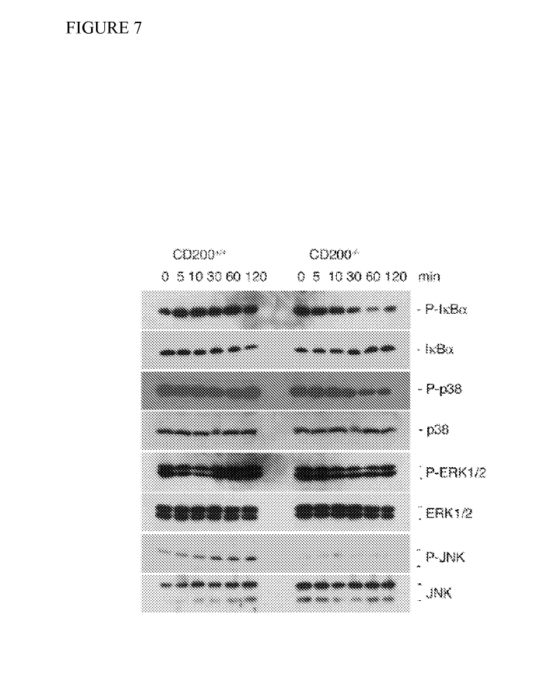

FIG. 8: The CD200-CD200R axis is required for osteoclast fusion/multinucleation. Bone marrow-derived macrophages from six-week-old wild-type mice were cultured in the presence of M-CSF (30 ng/ml) and RANKL (50 ng/ml) with or without the recombinant extracellular domain of CD200 (rCD200e; 100 ng/ml). rCD200e allowed the differentiation of osteoclasts in macrophages that lacked CD200 (SD; n=3). Bone marrow macrophages isolated from CD200-deficient and wild-type mice were cultured in the presence of M-CSF (5 ng/ml) for 12-18 h. Non-adherent cells were cultured for a further two days in the presence of M-CSF (30 ng/ml), starved for 2 h, and then treated with RANKL (50 ng/ml) with or without rCD200e (0.5 ug/ml) for 30 min. The cells were then subjected to Western blotting analysis with the indicated antibodies against IkBα and JNK and their phosphorylated forms. The addition of rCD200e restored the activation of JNK and of IkBα.

FIG. 9: Bone marrow-derived macrophages from six-week-old wild-type mice were cultured in the presence of M-CSF (30 ng/ml) and RANKL (100 ng/ml) with or without the recombinant extracellular domain of the CD200 receptor (rCD200Re). rCD200Re blocked the fusion of macrophages (SD; n=5). Bone marrow-derived macrophages from six-week-old wild-type mice were cultured in the presence of M-CSF (30 ng/ml) for two days prior to being transduced with the retroviral vector MigR1, which encoded, or not, short hairpin RNAs designed after the CD200R1 cDNA. A construct encoding random (rdm) oligonucleotides was used as a negative control. Each of the three targeting retroviral constructs, namely shRNAi1, shRNAi2 and shRNAi3, abolished the expression of CD200R1 and prevented the formation of multinucleate osteoclasts.

FIG. 10: Bone density increased in the absence of CD200. A, Distal femurs from six-month-old male and female CD200-deficient mice exhibited an increased trabecular density. CD200-deficient males also had greater subcortical contents than wild-type males (pQCT; n=8). B, The absence of CD200 did not prevent bone loss in response to ovariectomy. Two-month-old female CD200-deficient and wild-type mice were used as controls or they were subjected to sham operation or to ovariectomy (n=8; SD).

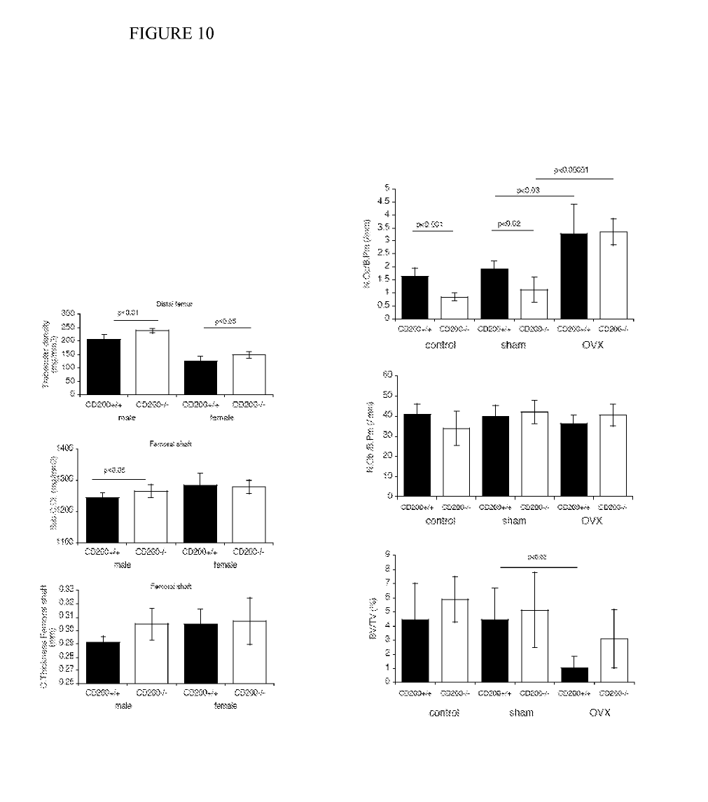

### DETAILED DESCRIPTION OF THE INVENTION

The term “patient” includes both human and non-human mammals.

The terms “treating” or “treatment” mean the treatment of a disease-state in a patient, and include:

(i) preventing the disease-state from occurring in a patient, in particular, when such patient is genetically or otherwise predisposed to the disease-state but has not yet been diagnosed as having it;
(ii) inhibiting or ameliorating the disease-state in a patient, i.e., arresting or slowing its development; or
(iii) relieving the disease-state in a patient, i.e., causing regression or cure of the disease-state.

Putative compounds as referred to herein include, for example, compounds that are products of rational drug design, natural products and compounds having partially defined signal transduction regulatory properties. A putative compound can be a protein-based compound, a carbohydrate-based compound, a lipid-based compound, a nucleic acid-based compound, a natural organic compound, a synthetically derived organic compound, an anti-idiotypic antibody and/or catalytic antibody, or fragments thereof. A putative regulatory compound can be obtained, for example, from libraries of natural or synthetic compounds, in particular from chemical or combinatorial libraries (i.e., libraries of compounds that differ in sequence or size but that have the same building blocks; see for example, U.S. Pat. Nos. 5,010,175 and 5,266,684 of Rutter and Santi, which are incorporated herein by reference in their entirety) or by rational drug design.

A suitable amount of putative regulatory compound(s) suspended in culture medium is added to the cells that is sufficient to regulate the activity of a CD200, CD200R protein in a cell such that the regulation is detectable using a known detection methods. A preferred amount of putative regulatory compound(s) comprises between about 1 nM to about 10 mM of putative regulatory compound(s) per well of a 96-well plate. The cells are allowed to incubate for a suitable length of time to allow the putative regulatory compound to enter a cell and interact with the target protein. A preferred incubation time is between about 1 minute to about 48 hours.

The technology for producing monoclonal antibodies is well known. In general, an immortal cell line (typically myeloma cells) is fused to lymphocytes (typically splenocytes) from a mammal immunized with whole cells expressing a given antigen, e.g., CD200, CD200R, and the culture supernatants of the resulting hybridoma cells are screened for antibodies against the antigen. See, generally, Kohler et at., 1975, Nature 265: 295-497, “Continuous Cultures of Fused Cells Secreting Antibody of Predefined Specificity”.

Immunization may be accomplished using standard procedures. The unit dose and immunization regimen depend on the species of mammal immunized, its immune status, the body weight of the mammal, etc. Typically, the immunized mammals are bled and the serum from each blood sample is assayed for particular antibodies using appropriate screening assays. For example, anti-integrin antibodies may be identified by immunoprecipitation of 125I-labeled cell lysates from integrin-expressing cells. Antibodies, including for example, anti-CD200, CD200R antibodies, may also be identified by flow cytometry, e.g., by measuring fluorescent staining of antibody-expressing cells incubated with an antibody believed to recognize CD200, CD200R molecules. The lymphocytes used in the production of hybridoma cells typically are isolated from immunized mammals whose sera have already tested positive for the presence of anti-CD200, CD200R antibodies using such screening assays.

Typically, the immortal cell line (e.g., a myeloma cell line) is derived from the same mammalian species as the lymphocytes. Preferred immortal cell lines are mouse myeloma cell lines that are sensitive to culture medium containing hypoxanthine, aminopterin and thymidine (“HAT medium”). Typically, HAT-sensitive mouse myeloma cells are fused to mouse splenocytes using 1500 molecular weight polyethylene glycol (“PEG 1500”). Hybridoma cells resulting from the fusion are then selected using HAT medium, which kills unfused and unproductively fused myeloma cells (unfused splenocytes die after several days because they are not transformed). Hybridomas producing a desired antibody are detected by screening the hybridoma culture supernatants. For example, hybridomas prepared to produce anti-CD200, CD200R antibodies may be screened by testing the hybridoma culture supernatant for secreted antibodies having the ability to bind to a recombinant CD200, CD200R—expressing cell line.

To produce antibody homologs which are within the scope of the invention, including for example, anti-CD200, CD200R antibody homologs, that are intact immunoglobulins, hybridoma cells that tested positive in such screening assays were cultured in a nutrient medium under conditions and for a time sufficient to allow the hybridoma cells to secrete the monoclonal antibodies into the culture medium. Tissue culture techniques and culture media suitable for hybridoma cells are well known. The conditioned hybridoma culture supernatant may be collected and the anti-CD200, CD200R antibodies optionally further purified by well-known methods.

Alternatively, the desired antibody may be produced by injecting the hybridoma cells into the peritoneal cavity of an unimmunized mouse. The hybridoma cells proliferate in the peritoneal cavity, secreting the antibody which accumulates as ascites fluid. The antibody may be harvested by withdrawing the ascites fluid from the peritoneal cavity with a syringe.

Fully human monoclonal antibody homologs against, for example CD200, CD200R, are another preferred binding agent which may block antigens in the method of the invention. In their intact form these may be prepared using in vitro-primed human splenocytes, as described by Boerner et al., 1991, J. Immunol. 147:86-95, “Production of Antigen-specific Human Monoclonal Antibodies from In Vitro-Primed Human Splenocytes”.

Alternatively, they may be prepared by repertoire cloning as described by Persson et al., 1991, Proc. Nat. Acad. Sci. USA 88: 2432-2436, “Generation of diverse high-affinity human monoclonal antibodies by repertoire cloning” and Huang and Stollar, 1991, J. Immunol. Methods 141: 227-236, “Construction of representative immunoglobulin variable region CDNA libraries from human peripheral blood lymphocytes without in vitro stimulation”. U.S. Pat. No. 5,798,230 (Aug. 25, 1998, “Process for the preparation of human monoclonal antibodies and their use”) describes preparation of human monoclonal antibodies from human B cells. According to this process, human antibody-producing B cells are immortalized by infection with an Epstein-Barr virus, or a derivative thereof, that expresses Epstein-Barr virus nuclear antigen 2 (EBNA2). EBNA2 function, which is required for immortalization, is subsequently shut off, which results in an increase in antibody production.

In yet another method for producing fully human antibodies, U.S. Pat. No. 5,789,650 (Aug. 4, 1998, “Transgenic non-human animals for producing heterologous antibodies”) describes transgenic non-human animals capable of producing heterologous antibodies and transgenic non-human animals having inactivated endogenous immunoglobulin genes. Endogenous immunoglobulin genes are suppressed by antisense polynucleotides and/or by antiserum directed against endogenous immunoglobulins. Heterologous antibodies are encoded by immunoglobulin genes not normally found in the genome of that species of non-human animal. One or more transgenes containing sequences of unrearranged heterologous human immunoglobulin heavy chains are introduced into a non-human animal thereby forming a transgenic animal capable of functionally rearranging transgenic immunoglobulin sequences and producing a repertoire of antibodies of various isotypes encoded by human immunoglobulin genes. Such heterologous human antibodies are produced in B-cells which are thereafter immortalized, e.g., by fusing with an immortalizing cell line such as a myeloma or by manipulating such B-cells by other techniques to perpetuate a cell line capable of producing a monoclonal heterologous, fully human antibody homolog.

### Expression of CD200 De Novo in Macrophages at the Onset of Fusion

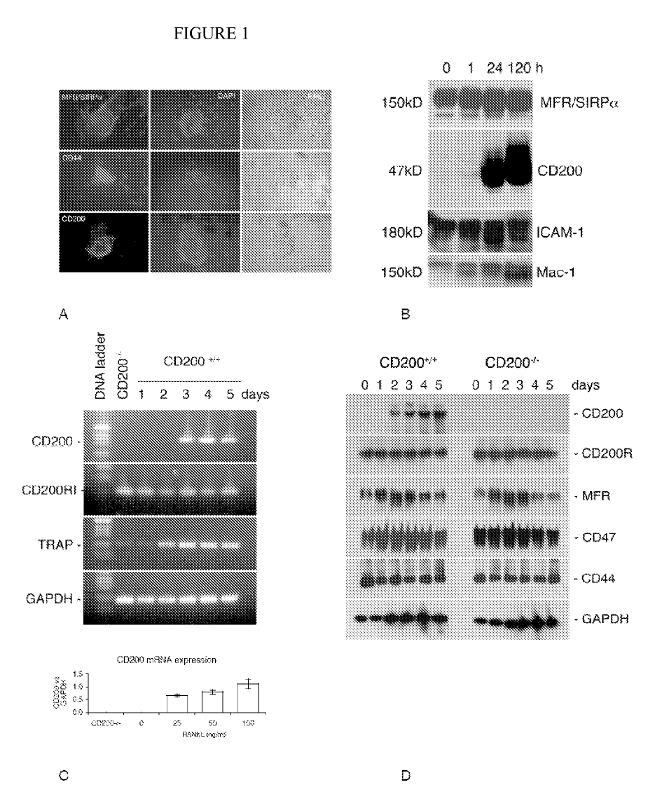

To identify novel components of the machinery of macrophage fusion, we submitted fusing alveolar macrophages from rats to genome-wide microarray analysis. Such macrophages provide an efficient and homogeneous model system for studies of macrophage fusion (Saginario, 1995, 1998; Sterling, 1998; Han, 2000; see Vignery, 2005 for a review) since they are “naïve” and fuse spontaneously in vitro, when plated confluently, without the addition of cytokines Barely any transcripts encoding CD200 (accession # X01785) were detected in freshly isolated macrophages, but the levels of transcripts were 0.6+/−1.4, 34.9+/−7.2 and 61.6+/−23.4 times higher than those in freshly isolated cells 1 h, 24 h and 120 h after plating, respectively (mean+/−SD; n=3). To confirm the cell-surface expression of CD200, we reacted multinucleated alveolar macrophages with a monoclonal antibody raised against the extracellular domain of CD200. In parallel, we subjected fusing alveolar macrophages to Western blotting analysis at different times. We used antibodies directed against MFR/SIRPα as a control because the expression of this protein is induced at the onset of macrophage fusion (Saginario, 1995). Our results confirmed the strong and de novo expression of CD200 as early as 24 h after plating (FIGS. 1A, B). However, unlike MFR/SIRPα, which is expressed in mononucleate macrophages, CD200 was not expressed in mononucleate macrophages (FIG. 1A).

To investigate whether CD200 was also expressed in osteoclasts, we cultured mouse bone marrow macrophages in the presence of M-CSF (30 ng/ml) and RANKL (50 ng/ml) for five days to generate osteoclasts (Li et al., 2005). No transcripts encoding CD200 were detected in macrophages, but strong expression of such transcripts was induced by RANKL as early as day 2. Moreover, the induction of expression of CD200 was dependent on the dose of RANKL (FIG. 1C). By contrast, the expression of CD200R was clearly constitutive (FIG. 1D). Moreover, while MFR/SIRPα, CD47 and CD44 were expressed in osteoclasts during their differentiation, the levels of these proteins were unaffected by disruption of the expression of CD200 since osteoclasts from mice deficient in CD200 expressed similar levels of these proteins. This observation suggests that the expression of these fusion molecules is regulated by a mechanism that is independent of CD200.

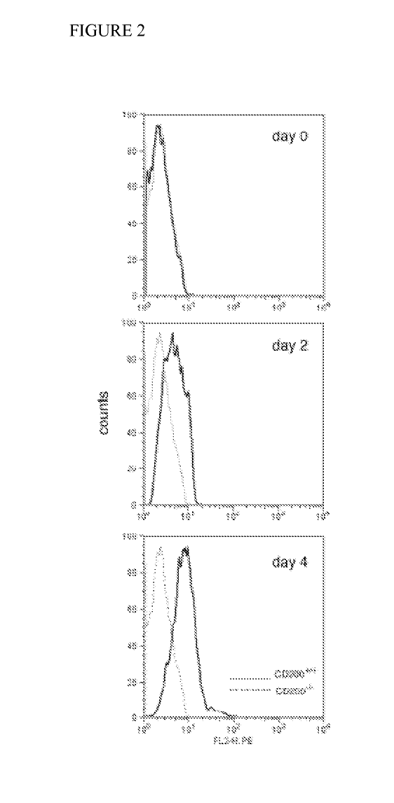

To confirm that CD200 was expressed on the surface of osteoclasts, we cultured bone macrophages as described previously, reacted them at different times with a monoclonal antibody that recognized the extracellular domain of CD200 and subjected them to flow-cytometric analysis. The results, shown in FIG. 2, confirm the strong and de novo cell-surface expression of CD200 at the onset of osteoclast fusion/multinucleation.

Together, our results indicate that CD200 might be a previously unrecognized component of the macrophage fusion machinery. Therefore, we postulated that the deletion of CD200 would affect differentiation of osteoclasts, and as a result, the development and/or the remodeling of bone.

CD200-Deficient Mice Had Higher Bone Density and Fewer Osteoclasts than Wild-Type Mice

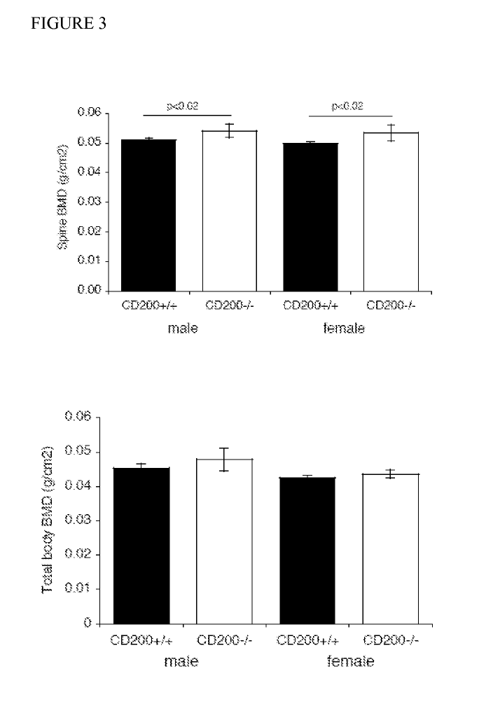

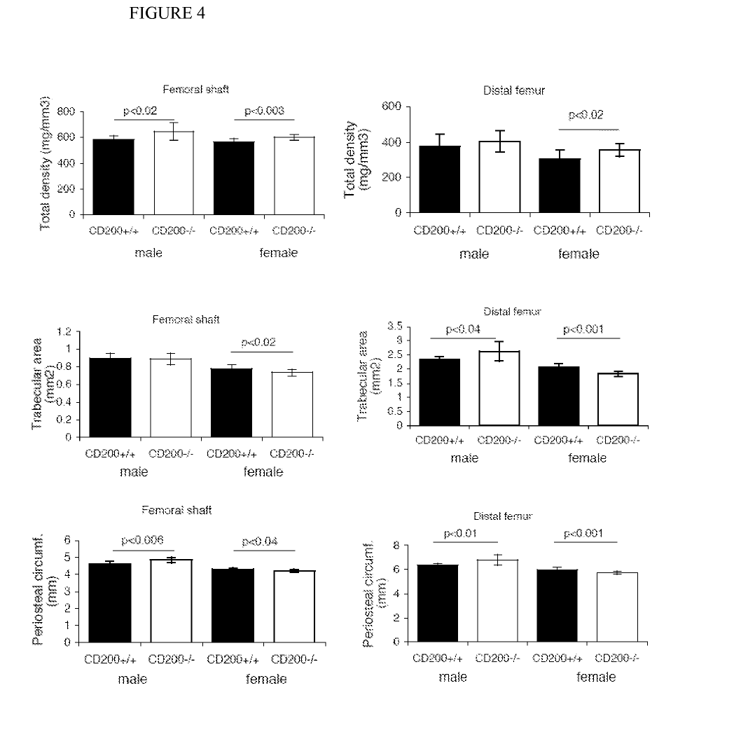

We subjected two-month-old male and female CD200^{-/-} and wild-type mice to DEXA analysis (see “Material and Methods”). As we had predicted, both male and female CD200^{-/-} mice had higher spinal bone densities than corresponding wild-type mice (FIG. 3). Peripheral quantitative tomography (pQCT) analysis of the femurs from these mice revealed that CD200 deficiency was associated with an increase in the total density of the shaft in both males and females, and of the distal femur in females, as compared to age- and sex-matched wild-type mice (FIG. 4). In CD200-deficient female mice, there was an increase in the trabecular area of the shaft and the distal part of the femur while in CD200-deficient male mice, there was an increase in the trabecular area of the distal femur only, as compared with the respective wild-type mice. In CD200-deficient male mice there was an increase, and in CD200-deficient female mice there was a decrease, in periosteal circumference, in both the shaft and the distal femur, as compared to corresponding wild-type mice. It appeared, therefore, that CD200 deficiency has lead to the enhanced accumulation of bone, with the shapes and size of bones being altered in a gender-specific manner.

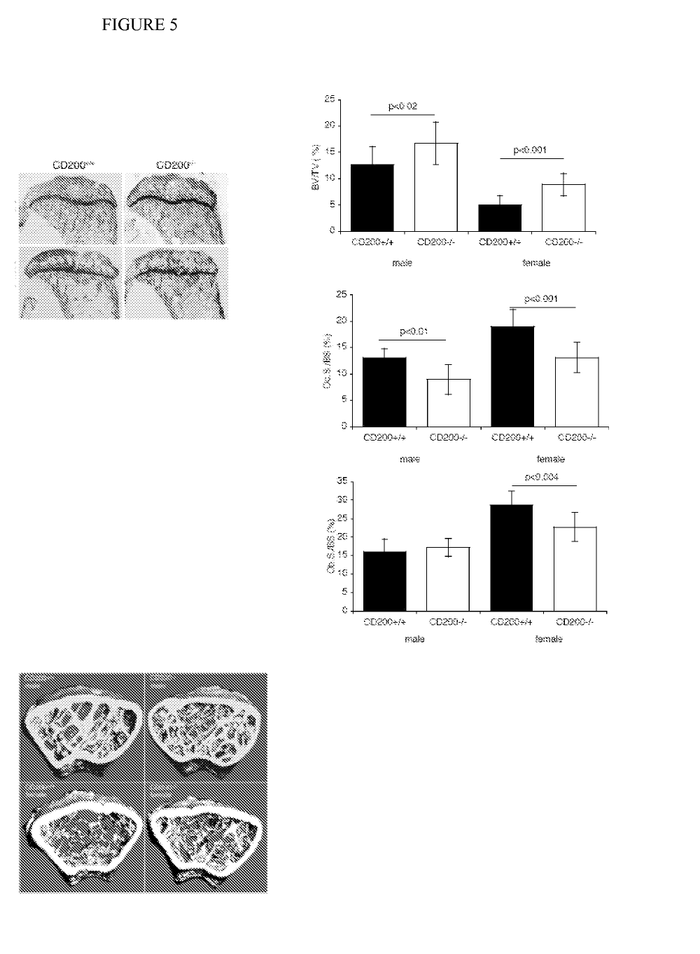

To analyze the cellular mechanisms by which the deletion of CD200 augments total bone density, we subjected the distal femurs from CD200-deficient and age- and sex-matched wild-type mice to histomorphometric analysis. Our results confirmed that CD200-deficient mice, both males and females, had an increase in trabecular bone volume when compared to the wild types (FIG. 5). We also found a decrease in the relative bone surface area that was occupied by osteoclasts in both male and female CD200-deficient mice. To our surprise, despite the increase in bone density in CD200-deficient female mice, we found a decrease in the relative surface area of bone that was covered by osteoblasts (FIG. 5). This result suggested that it was, indeed, the osteoclast that were responsible for the higher bone volume in CD200-deficient mice.

To determine whether the increase in bone volume persisted with aging, we subjected the distal femurs from both CD200-deficient and wild type 6-month-old mice to microCT analysis. Both male and female CD200-deficient mice had more trabecular bone than the corresponding wild types (FIG. 5). This observation was supported by pQCT analysis of the same bones, which showed that trabecular density was higher in the CD200-deficient mice than in the corresponding wild types (data not shown).

### The Absence of CD200 Impaired the Differentiation of Osteoclasts but not of Osteoblasts

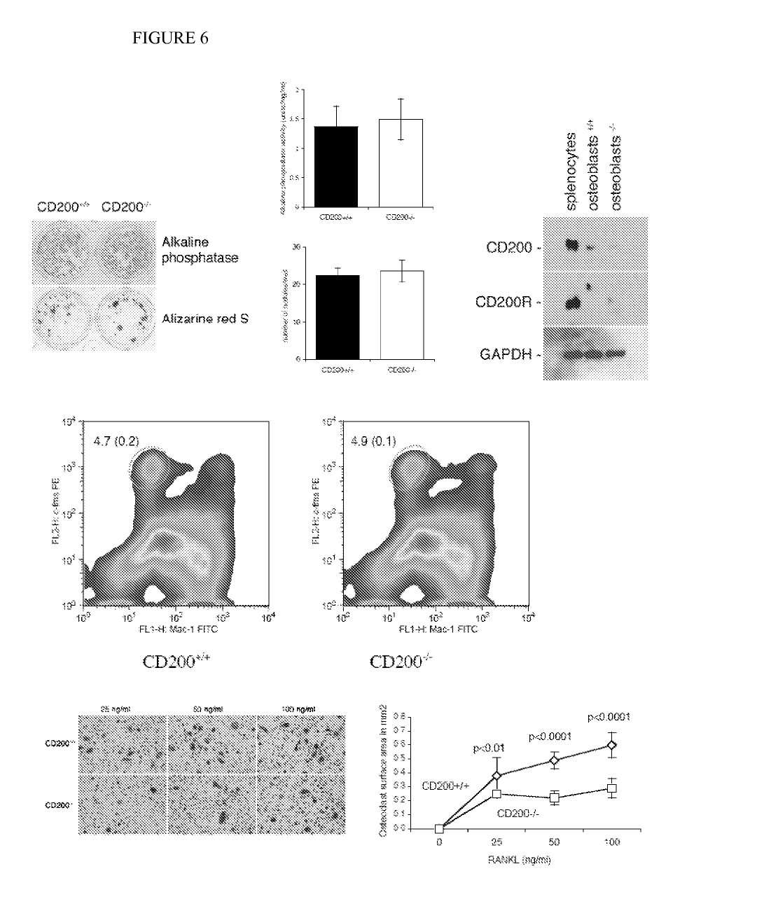

To determine whether the absence of CD00 might affect the differentiation of osteoblasts, we cultured bone marrow cells from CD200-deficient and wild-type mice for 9 days in the presence of ascorbic acid (50 μg/ml) and 13-glycerophosphate (10 mM) and the we compared their respective alkaline phosphatase activities and their abilities to form bone-like nodules. The absence of CD200 had no effect on alkaline phosphatase activity and on the formation of nodules by osteoblasts (FIG. 6). However, Western blotting analysis of osteoblasts confirmed the relatively low-level expression of CD200 (Lee et al., 2006) and the absence of CD200R. These data suggested that the increase in bone volume could not be attributed to osteoblasts.

The decrease in the number of osteoclasts seen in vivo in CD200-deficient mice might result from a decrease in the number of osteoclast precursor cells or from a defect in osteoclast formation. To examine these possibilities, we subjected freshly isolated bone marrow cells to flow-cytometric analysis using surface markers expressed by pre-osteoclasts (c-Fms⁺/c-Kit⁺/Mac1^{low}; Arai et al., 1999; Jimi et al., 2004). The percentage of precursor cells relative to the total number of bone marrow cells was similar in CD200^{+/+} and CD200^{-/-} mice (FIG. 6). We then compared the rates of osteoclastogenesis in vitro in CD200 ‘/’ and CD200^{-/-} mice. We cultured mouse bone marrow macrophages in the presence of M-CSF (30 ng/ml) and increasing concentrations of RANKL for five days to generate osteoclasts. The absence of CD200 resulted in a dose-dependent decrease in the number of osteoclasts and in the surface area covered by osteoclasts (FIG. 6). These data strongly supported our hypothesis that the increase in bone volume resulted from a defect in the formation of osteoclasts.

Since RANKL, which activates the NF-kB and MAP kinase signaling pathways that operate downstream of RANK, is essential for osteoclastogenesis, we next asked whether a deficiency in CD200 might affect signaling downstream of RANK. We cultured bone marrow cells from CD200-deficient and wild-type mice in the presence of M-CSF (30 ng/ml) for two days, then, after starving them for two hours, we treated them with RANKL (50 ng/ml) up to 2 hours and, finally, we subjected them to Western blotting analysis with phosphorylated form-specific and control antibodies directed against IkBα, p38, ERK1/2 and JNK. While the extent of activation of IkBα decreased slightly with time, activation of JNK was almost completely abolished in cells that lacked CD200. These results revealed that the absence of CD200 attenuated the transduction of signals downstream of RANK and suggested that the CD200-CD200R interaction might play a role in this signaling pathway and in the formation of osteoclasts.

### The CD200-CD200R Axis is a Novel Component of the Fusion Machinery

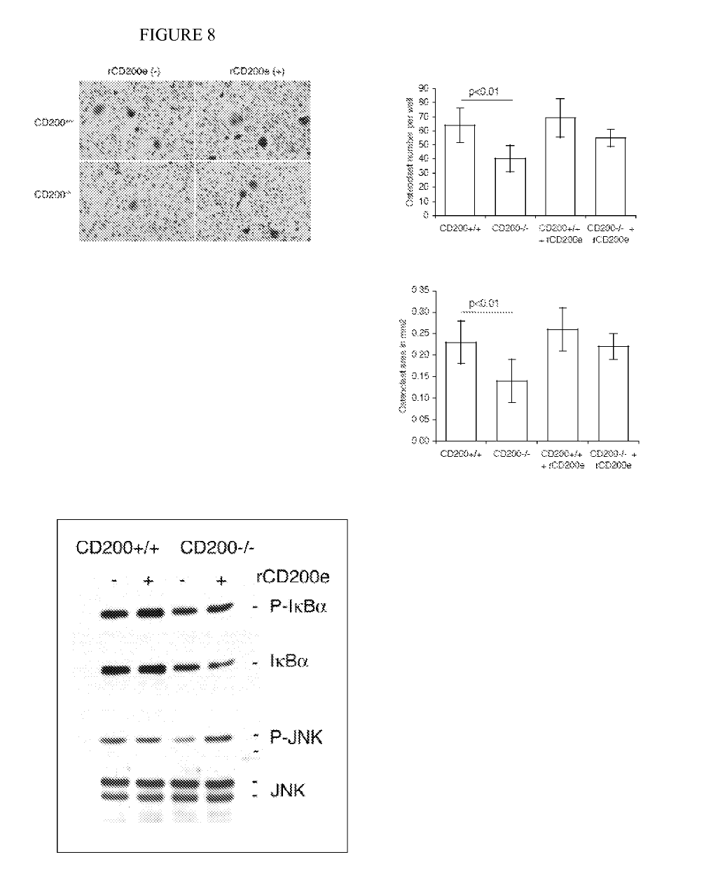

To address the putative role of the CD200-CD200R axis in the fusion of macrophages, we used several complementary strategies. First, we asked whether exogenous CD200 could rescue the differentiation of osteoclasts in vitro in cells that lack CD200. We generated a soluble recombinant protein that included the extracellular domain of mouse CD200 (rCD200e). We cultured bone marrow cells isolated from CD200-deficient and wild-type mice in the presence of M-CSF (30 ng/ml), RANKL (50 ng/ml), and rCD200e (0.5 ug/ml). The addition of rCD200e rescued the differentiation of CD200-deficient osteoclasts (FIG. 8). We next asked whether rCD200e-induced fusion resulted from the activation of JNK and IkB activation, which is suppressed in the absence of CD200. We cultured bone marrow cells from CD200-deficient and wild-type mice in the presence of M-CSF (5 ng/ml) for two days. After starving them for two hours, we treated them for 30 minutes with RANKL (50 ng/ml), in the presence and in the absence of rCD200e (0.5 ug/ml) and, finally, we subjected them to Western blotting as described above. The addition of rCD200e restored the activation of IkBα and JNK, supporting a role for CD200 in the differentiation of osteoclasts via the CD200R-mediated activation of IkBα and JNK (FIG. 8).

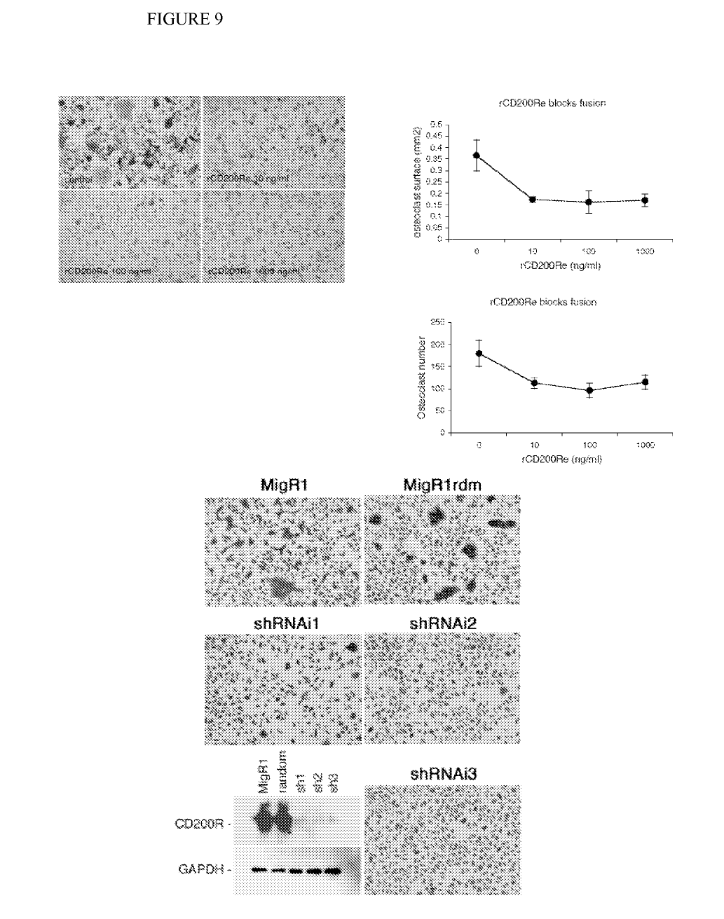

We postulated next that, if the CD200-CD200R interaction plays a role in fusion, interference with this interaction should block fusion. We engineered a soluble mouse recombinant protein that included the extracellular domain of CD200R (rCD200Re). We cultured bone marrow cells from wild-type mice in the presence of M-CSF (30 ng/ml) and RANKL (50 ng/ml) in the absence and in the presence of rCD200Re (10-1,000 ng/ml). As anticipated, osteoclastogenesis was blocked in the presence of rCD200Re (FIG. 9). In addition to CD200R, also known as CD200R1, mice express CD200R2, CD200R3 and CD200R4 (Wright et al., 2003). We found that mouse osteoclasts only expressed transcripts that encoded CD200R1 and CD200R4 (data not shown). To date, the functions of these additional receptors (CD200R2, R3 and R4) remain unclear. However, it has been demonstrated that CD200 only binds to and activates CD200R1 (Hatherley et al., 2005).

Finally, to investigate the role of CD200R in fusion more directly, we attempted to silence the expression of this receptor in fusing macrophages by RNA interference (RNAi) with short hairpin RNA (shRNA). We generated three retrovirus-based shRNA constructs that targeted CD200R1 (shRNAi1, shRNAi2 and shRNAi3), as well as a construct that encoded random sequences (MigR1rdm). We transduced bone marrow macrophages isolated from wild-type mice with these constructs, as well as the empty vector (MigR1). Each one of the shRNA constructs (shRNAi1, shRNAi2 and shRNAi3) interfered with the expression of CD200R and prevented the fusion of osteoclasts (FIG. 9). By contrast, neither MigR1 nor MigR1rdm affected the expression of CD200R and the differentiation of osteoclasts. Together, these results confirmed the proposed central role for the CD200-CD200R axis in the fusion of macrophages and in osteoclastogenesis.

The CD200-CD200R axis appears to be a novel and central player in the fusion and/or multinucleation of macrophages, which is required for the differentiation of osteoclasts, and the regulation of bone mass. While our results confirm that mononucleate macrophages do not express CD200, they reveal that their fusion is accompanied by strong and de novo expression of CD200. Not only is the expression of CD200 abruptly induced in fusing osteoclasts, but absence of CD200 impairs osteoclastogenesis, with a subsequent increase in bone volume and, hence, a mild form of osteopetrosis.

Our analysis of the number of bone marrow macrophages/osteoclast precursor cells as a percentage of the total number of bone marrow cells, which was similar in CD200-deficient and wild-type mice, suggests that the CD200-CD200R axis does not control the differentiation of pre-monocytes (Fumio Arai et al, 1999; Eijiro Jimi et al, 2004 for facs analysis). This is in contrast with the numbers of splenic and mesenteric lymph node macrophages, which are elevated in mice that lack CD200 (Hoek et al. 2000). It is possible that from bone marrow are less differentiated than those from lymphoid organs, which might express low levels of CD200. Nevertheless, the decreases in the numbers of osteoclasts in CD200-deficient mice cannot be attributed to decreases in numbers of precursor cells.

Both CD200 and CD200R, resembling CD4, the receptor for HIV, and Izumo (Inoue et al., 2005), the sperm-fusion protein, belong to IgSF, a resemblance that suggests some commonality in the mechanics of cell fusion. In addition, genes for CD200-like proteins have been identified in the genomes of some, but not all, members of families of double-stranded DNA viruses, such as Poxviruses, Herpesviruses, and Adenoviruses (Chung et al. 2002; Foster-Cuevas et al. 2004). Moreover, the product of the K14 gene of Kaposi's sarcoma-associated Herpesvirus is a ligand for CD200R (Chung, 2002; Foster-Cuevas, 2004). Similarly, M141R is a cell-surface protein encoded by Myxoma virus with significant homology at the amino acid level to CD200, and it is required for the full pathogenesis of Myxoma virus in the European rabbit (Cameron et al. 2005). Most importantly, both CD200 and its viral homologs activate the CD200R to down regulate basophiles (HHV-8; Shiratori, 2005) and macrophages (HHV-8 and M141R; Foster-Cuevas, 2004; Cameron, 2005) function. Hence, as might be the case for CD47, which is homologous to proteins encoded by Vaccinia and Myxoma virus (Parkinson et al., 1995; Cameron et al., 2005), viruses might have “stolen” CD200 to allow them to evade the immune response and to fuse with and infect cells.

While the CD200-CD200R axis plays an inhibitory role in the immune system (see Minas and Liversidge, 2006, for a review), it appears to play an activating role in macrophage fusion since the absence of CD200 slows down the differentiation of osteoclasts. Since it has been proposed that the MFR/SIRPα-CD47 axis plays an activating role in the formation of osteoclasts, it is possible that these two axes work in tandem to secure the differentiation of osteoclasts. Mice that lack both CD47 and CD200 might provide a model to answer this question. In addition, we cannot exclude the possibility that CD200 and its receptor associate both in cis and in trans via their amino-terminal domains, since the fusing partners are both macrophages. Indeed, it will be of interest to determine whether downstream signaling is differentially activated in cis or in trans in future studies.

The fact that a defect in osteoclastogenesis in CD200-deficient mice results from a defect in activation downstream of RANK suggests possible cross-talk between CD200R and RANK. We should note, however, that while the absence of CD200 slows down osteoclastogenesis, it does not prevent the expression of MFR/SIRPα and CD44, which are candidate members of the fusion machinery in macrophages. It remains to be determined whether the absence of CD200 affects the expression of DC-STAMP, the most recently identified component of macrophage fusion machinery (Yagi, 2005; Vignery, 2005). Together, our results suggest that the machinery for macrophage fusion involves multiple and, possibly, redundant molecules.

The absence of CD200 increased bone mass and the soluble recombinant extracellular domain of CD200R blocked macrophage fusion in vitro. Thus, CD200 and its receptor might be novel targets in efforts to prevent bone loss. However, even though osteoblasts in culture express low levels of CD200 and the absence of CD200 does not affect their differentiation in vitro, we cannot exclude a possible role for CD200 in these cells in vivo. Further studies involving the treatment of animal models with the soluble recombinant extracellular domain of CD200R will help clarify this issue.

### Experimental Section

Animals. CD200^{-/-} mice were produced by homologous recombination as described previously (Hoek et al, 2000). Mice were screened by PCR using the CD200^{+/+} forward primer 5′-gtagaagatccctgcatccatcag-3′ and reverse primer 5′-gcccagaaaacatggtcacctac-3′, which generate PCR products of 1000 bases for wild type and 1250 bases for CD200-deficient mice. Animals were housed and bred at the Yale Animal Care facility, under sterile conditions reserved for immuno-deficient mice, which include autoclaved cages and food, as well as changing of cages in a clean-air cabinet/change station using sterile techniques. Mice whose bones were subjected to histomorphometric analysis received two i.p. injections of calcein (3 ug/g body weight; Merck, Darmstadt, Germany) on days 1 and 6 before sacrifice. The Yale Animal Care and Use Committee approved all experiments.

### Bone Radiography

Excised femurs were subjected to X-ray using a MX-20 (Faxitron X-ray Corporation, Wheeling, Ill.) at 30 kV for 3 seconds. X-rays were scanned using an Epson Perfection 4870.

Computed Tomography on a Microscale (microCT)

The proximal tibiae from 6-month-old male CD200^{-/-} and CD200^{+/+} mice were scanned with a microCT scanner (□□□□□CT 40; Scanco, Bassersdorf, Switzerland) with a 2,048×2,048 matrix and isotropic resolution of 9 um³ with 12 um voxel size, three-dimensional trabecular measurements in the secondary spongiosa were made directly, as previously described (Li et al, 2005).

### Bone Densitometry

Bone density was determined as described previously (Ballica et al, 1998) by peripheral quantitative computed tomography (pQCT) with a Stratec scanner model XCT 960M (Norland Medical Systems, Fort Atkinson, Wis.). Routine calibration was performed daily with a defined standard that contained hydroxyapatite crystals embedded in lucite, provided by Norland Medical Systems. We scanned 1-mm-thick slices located at a distance of 3 mm, proximally, from the distal end of distal femoral metaphyses. The voxel size was set at 0.15 mm. Scans were analyzed with a software program supplied by the manufacturer (XMICE, version 5.1). Bone density and geometric parameters were estimated by Loop analysis. The low- and high-density threshold settings were 1,300 and 2,000, respectively. Separation of soft tissue from the outer edge of bone was achieved using contour mode 1. Cortical (high bone density) and trabecular (low bone density) bone were separated to obtain trabecular data using peel mode 3. Cortical and trabecular bone were separated to obtain cortical data using cortical mode 1.

### Histomorphometry

Tibiae from CD200^{+/+} and CD200^{-/-} mice were dehydrated in a graded ethanol series and embedded without decalcification in methylmethacrylate, as we described previously (Baron et al, 1982). Longitudinal sections were cut with an Autocut™ microtome with a tungsten carbide blade (Jung, Reichert, Germany). Four-um-thick sections were stained with toluidine blue (pH 3.7) and subjected to static histomorphometric analysis; while 8-um-thick sections were mounted, unstained, for dynamic histomorphometric analysis, which was performed at a constant distance from the growth plate (including trabecular bone), with an image analysis system (Osteomeasure™; Osteometrics, Atlanta, Ga.). The measured parameters included the bone volume relative to the total volume (BV/TV); the rate of bone formation (BFR/BV), which takes into account the mineral apposition rate; the number of osteoclasts per active resorption perimeter (N.Oc/B.Pm); the number of osteoblasts per active formation perimeter (N.Ob/B.Pm); and the osteoid volume relative to bone volume (OV/BV).

### Reagents

Recombinant mouse RANKL and M-CSF were obtained from R&D Systems (Minneapolis, Minn.). A mouse monoclonal antibody directed against rat CD200 and rat monoclonal antibodies directed against mouse CD200 and CD200R were purchased from Serotec (Raleigh, N.C.). A polyclonal antibody directed against the intracellular domain of MFR was published previously (Han, 2000). Rabbit polyclonal antibodies directed against p38, phosphorylated-p38 (P-p38), ERK1/2, P-ERK1/2, JNK, and mouse monoclonal antibodies directed against IkB, P-IkB and P-JNK were obtained from Cell Signaling (Beverly, Mass.). A monoclonal antibody directed against mouse CD44 was obtained from BD Bioscience (Franklin Lakes, N.J.). A mouse monoclonal antibody directed against GAPDH was purchased from Novus Biologicals, Inc. (Littleton, Colo.). Horseradish peroxidase-conjugated F(ab′)₂ directed against rabbit and mouse IgG were purchased from Jackson ImmunoResearch (West Grove, Pa.). Rat anti-mouse monoclonal antibodies used for flow cytometry included anti-Mac-1 (CD-11b) conjugated to fluorescein (Mac1-FITC; M1/70; PharMingen, San Diego, Calif.), anti-c-fms conjugated to phycoreythrin (c-fms-PE) and anti-c-Kit conjugated to allophycocyanin (c-kit-APC; eBioscience, San Diego, Calif.). Secondary antibody anti-rat IgG2a conjugated to FITC was purchased from PharMingen. All supplies and reagents for tissue culture were endotoxin-free. Some bone marrow cells were treated with polymyxin B sulfate for 24 h to avoid the effects of the endotoxin prior to treatment.

### Bone Marrow Macrophages and Osteoclasts

Bone marrow cells from six- to twelve-week-old CD200^{-/-} and CD200 mice were plated in 10 cm dishes and cultured in a-MEM (Life Technologies, Grand Island, N.Y.) supplemented with 10% FBS in the presence of M-CSF (5 ng/m) (1×10⁷ cells/10 cm dish) for 12-18 h. Non-adherent cells were harvested and cultured with M-CSF (30 ng/ml) in 10 cm dishes, at the same density as before, for an additional 48 h. Floating cells were removed and attached cells, which were tartrate-resistant acid-phosphatase positive (TRAP⁺) macrophages were used as osteoclast precursors (Li et al., 2005). To generate osteoclasts, we cultured bone marrow macrophages in the presence of RANKL (50 ng/ml) and M-CSF (30 ng/ml) or a 30% (v/v) dilution of the supernatant from a culture of L929 cells, in 96-well, 24-well or 60 mm dishes at a density of 0.5×10⁶ cells/ml.

### Western Blotting Analysis

Cultured cells were lysed directly in non-denaturing RIPA buffer (150 mM NaCl, 20 mM Tris, pH 7.5, 1% NP40, 5 mM EDTA) supplemented with a cocktail of protease inhibitors (Complete Tablets, Roche Molecular Biochemicals) and phosphatase inhibitors cocktail 2 (Sigma, St Louis, Mich.). The lysates were sonicated, and the equivalent of 2×10⁵ cells per sample was loaded onto a 10% denaturing or non-denaturing acrylamide gel and run for 1-2 h. The proteins were transferred onto nitrocellulose membranes and blocked with 5% dry milk in T-PBS, and incubated with primary and secondary antibodies, sequentially. Finally, membranes were incubated with supersignal (ECL kit, Pierce Chemical Co., New York, N.Y.) and exposed to x-ray film.

### Generation of Osteoclasts Using Non-Adherent Bone Marrow Cells

Bone marrow cells from 6- to 8-wk-old CD200^{+/+} and CD200^{-/-} mice were plated in 10 cm dishes and cultured in α-MEM supplemented with 10% FBS in the presence of M-CSF (10 ng/m; 10⁷ cells/10-cm dish) for 12-18 h. Non-adherent cells were harvested and cultured with M-CSF (30 ng/ml) in 10 cm dishes, at the same density as before, for an additional 48 h. Floating cells were removed and attached cells were used as osteoclast precursor cells. To generate osteoclasts, bone marrow macrophages were cultured in the presence of RANKL (25-100 ng/ml) and M-CSF (30 ng/ml) or a 30% (vol/vol) dilution of the supernatant from a culture of L929 cells, in 96-well, 24-well, or 60 mm dishes at a density of 4×10⁵ cells/cm². TRAP-positive osteoclast-like multinucleated cells (with more than two nuclei) were subjected to histomorphometry.

### Osteoblast Alkaline Phosphatase (ALP) Assay (Mikihiko Morinobu et al, 2005)

Bone marrow cells from 6- to 8-wk-old CD200^{+/+} and CD200 mice were plated in 24-well plates (5×10⁶ cells/well) and cultured in α-MEM supplemented with 10% FBS, 50 μg/ml ascorbic acid, 10 mM B-glycerophosphate. Medium was changed every 3 days, and the cells were cultured for 9 days. The cells were rinsed twice with ice-cold PBS and scraped into 10 mM Tris-HCl containing 2 mM MgCl₂ and 0.05% Triton X-100, pH 8.2. Cell lysates were briefly sonicated on ice after two cycles of freezing and thawing. Aliquots of supernatants were subjected to ALP activity measurement (Sigma, St Louis, Mich.) according to manufacturer's instruction, and protein concentration was determined according to Bradford.

### Mineralized Nodule Formation Assay (Mikihiko Morinobu et al, 2005)

Bone marrow cells from 6- to 8-wk-old CD200^{-/-} and CD200^{+/+} mice were plated in 24-well plates (5×10⁶ cells/well) and cultured in α-MEM supplemented with 10% FBS, 50 μg/ml ascorbic acid, 10 mM B-glycerophosphate, and antibiotics (100 U/ml penicillin G, 100 μg/ml streptomycin sulfate). Medium was changed every 3 days, and the cells were cultured for 11 days. At the end of the culture, cells were fixed with 10% formalin/saline and stained for calcium with alizarin red S (Sigma, St Louis, Mich.) to identify mineralized bone nodules. The number of nodules per well was recorded.

### Flow Cytometry

Cells were stained with the first antibody, incubated for 30 min on ice, and washed twice with washing buffer (5% FCS/PBS). The secondary antibody was added, and the cells were incubated for 30 min on ice. After incubation, cells were washed twice with washing buffer and suspended in washing buffer for FACS analysis, which was performed using a FACS Calibur (BD Bioscience, Franklin Lakes, N.J.).

### Reverse Transcriptase Polymerase Chain Reaction (RT-PCR)

Total RNA was extracted in Trizol (Invitrogen, Carlsbad, Calif.) according to manufacturer instruction. First-strand cDNA was synthesized using 1 μg of the total RNA and Moloney murine leukemia virus reverse transcriptase. Primer pairs for PCR reactions were as follows: GAPDH forward, 5′-AAACCCATCACCATCTTCCA-3; reverse, 5′-GTGGTTCACACCCATCACAA-3′, generating a size product of 198 bases; TRAP forward, 5′-CAGCTGTCCTGGCTCAAAA-3′ reverse, 5′-ACATAGCCCACACCGTTCTC-3′, generating a size product of 218 bases; CD200, forward, 5′-AGTGGTGACCCAGGATGAA-3; reverse, 5′-TACTATGGGCTGTACATAG-3′, generating a size product of 337 bases; CD200R1 forward, 5′-AGGAGGATGAAATGCAGCCTTA-3′; reverse, 5′-TGCCTCCACCTTAGTCACAGTATC-3′, generating a size product of 103 bases. For CD200R2, CD200R3 and CD200R4, we used the primers described by Voehringer et al. (2004).

Amplification was performed at 21-25 cycles within a linear range. Each cycle was set at 94C for 30 s; 55C for 30 s; and 72C for 40 s in a 50-μl reaction mixture containing 0.5 μl of each cDNA, 200 mM of each primer, 0.2 mM of dNTP, and 1 U Taq DNA polymerase (Invitrogen, Carlsbad, Calif.). After amplification, 30 μl of each reaction mixture was subjected to electrophoresis to be analyzed on 1.2% agarose gel. The bands were visualized by ethidium bromide staining, and scanned by digital camera. For semi-quantitative PCR study, the illuminant value of CD200 bands versus GAPDH internal controls was measured by Kodak ID5 software.

Generation of the Soluble Extracellular Domain of CD200 (sCD200e) and CD200R (sCD200Re)

The extracellular domain of CD200 and CD200R was amplified from splenocyte cDNA by RT-PCR, using following primers: CD200 Forward, 5′-CCCAAGCTTGGG CAAGTGGAAGTGGTGACCC-3′; Reverse, 5′-CGGGATCCCGTGGAACTGAAAACCAAAATCCT-3′; CD200R Forward: 5′-CCCAAGCTTGGG ACTGATAAGAATCAAACAACACAGAAC-3′; Reverse: 5′-CGGGATCCCG GTATGGAATATATGGTCGTAATGATTG-3′. PCR products were subcloned into pSectag/Hygro vector (Invitrogen, Carlsbad, Calif.) HindIII and BamHI sites. The sequence of the recombinant DNA was verified by sequencing. Soluble CD200 (sCD200e) and sCD200Re constructs were transfected into 293T cells, and supernatant was harvested 4 days after transfection. sCD200e and sCD200Re proteins were purified by Ni-NTA agarose beads (Qiagen, Valencia, Calif.). The final elution of sCD200e and sCD200Re proteins was dialysed against 1×PBS using Slide-A-Lyser (Pierce), and sterilized using a 0.22 um syringe filter.

Short Hairpin RNA Interference (shRNAi)

We generated short hairpin RNAs (shRNA) to silence CD200R expression by PCR amplifying U6-Zeocin-shRNAi vector using the following primers:

universal forward primer
5′-gaAGATCTtcGATTTAGGTGACACTATAG

(underline letters denote BglII restriction site)

Reverse primer for SH1
5′-gGAATTCcAAAAAAACCAATCATTACGACCATATATTCCATACCAAT
ATGGAATATATGGTCGTAATGATTGGTTGTCGACGGTGTTTCGTCCTTTC
CACAA-3′;
Reverse primer for SH2
5′-gGAATTCcAAAAATGGGCCTCCACACCTGACCACAGTCCTGACCAAT
CAGGACTGTGGTCAGGTGTGGAGGCCCAGTCGACGGTGTTTCGTCCTTTC
CACAA-3′;
Reverse primer for SH3
5′-gGAATTCcAAAAAAAGCAGTATTAATCACATGGATAATAAAGCCAAC
TTTATTATCCATGTGATTAATACTGCTTGTCGACGGTGTTTCGTCCTTTC
CACAA-3′;
Reverse primer for scramble control
5′-gGAATTCcAAAAAGACAAGGATGTGACGCCTATACTCTCACTCCAAA
GTGAGAGTATAGGCGTCACATCCTTGTCGTCGACGGTGTTTCGTCCTTTC
CACAA-3′

(underline letters denote EcoRI restriction site for SH1, SH2, SH3 and scramble). PCR products were subcloned into MigRI-IRES-GFP retroviral vector, as previously described (Pear et al, 1998). Retroviruses were generated by transfecting shRNA constructs into GPG293 packaging cell line. Mouse bone-marrow derived macrophages were transduced with shRNA for 8 hours, 2 days after replating non adherent cells. Infected cells were then cultured in growth medium supplemented with 30 ng/ml M-CSF overnight. Infection efficiency was about 45-50%, and was monitored by GFP expression under U.V light. Infected cells were treated with 100 ng/ml RANKL for 3 days, and TRAP-positive osteoclasts were recorded.

### Statistical Analysis

Statistically significant differences among experimental groups were evaluated by the analysis of variances (Zar, 1984). The significance of mean changes was determined by an unpaired Student's two-tailed t-test, and significance was recognized when p<0.05.

### REFERENCES

Baron, R., Vignery, A., Neff, L., Silvergate, A., and Santa Maria, A. 1982. Processing of undecalcified bone specimens for bone histomorphometry. In Bone Histomorphometry: Techniques and Interpretation. R R Recker, H M Frost, Eds. CRC Press, Inc., Boca Raton, Fla. 13-35.
Vignery A. Osteoclasts and giant cells: macrophage-macrophage fusion mechanism. 2000. Int. J. Exp. Pathol. 81:291-304.
Vignery, A. Macrophage fusion: the making of osteoclasts and giant cells. Commentary, 2005. J. Exp. Med. 202: 337-340.
Vignery, A. Macrophage fusion: are somatic and cancer cells possible partners? 2005. Trends Cell Biol. 15:188-193.
Hernandez, L. D., Hoffman, L. R., Wolfsberg, T. G., and White, J. M. Virus-cell and cell-cell fusion. 1996. Annu Rev. Cell Dev. Biol. 12:627-661. Review
Dalgleish, A. G., Beverly, P., Clapham, P., Crawford, D., Greaves, M., and Weiss, R. The CD4 (T4) antigen is an essential component of the receptor for the AIDS retrovirus. 1984. Nature (London) 312:763-767.
Klatzmann, D., Champagne, E., Chamaret, S., Grust, J., Guetard, D., Hercent, T., Gluckmann, J. C., and Montagnier, L. T-lymphocyte T4 molecule behaves as the receptor for human retrovirus LAV. 1984. Nature (London). 312:767-768.
Saginario, C., Qian, H.-Y., and Vignery, A. Identification of an inducible surface molecule specific to fusing macrophages. 1995. Proc. Natl Acad. Sci, USA 92: 12210-12214.
Saginario, C., Sterling, H., Beckers, C., Kobayashi, R., Solimena, M., Ullu, E., and Vignery A. MFR, a putative receptor mediating the fusion of macrophages. 1998. Mol. Cell Biol. 18: 6213-6223.
Han, X., Sterling, H., Chen, Y., Saginario, C., Brown, E. J., Frazier, W. A., Lindberg, F. P., and Vignery, A. CD47, a ligand for MFR, participates in macrophage multinucleation. 2000. J. Biol. Chem. 275: 37984-37992.
Minas, K., and Liversidge, J. Is the CD200/CD200 receptor interaction more than just a myeloid cell inhibitory signal? 2006. Crit. Rev. Immunol. 26:213-230.
Sterling, H., Saginario, C., and Vignery, A. CD44 occupancy prevents the fusion of macrophages. 1998. J. Cell Biol. 143:837-847.
Cui, W., Zhang Ke, J., Zhang, Q., Ke, H.-Z., Chalouni, C., and Vignery, A. The intracellular domain of CD44 promotes the fusion of macrophages. 2006. Blood 107:796-805.
Arai, F., Miyamoto, T., Ohneda, O., Inada, T., Sudo, T., Brasel, K., Miyata, T., Anderson D. M., and Suda T. Commitment and differentiation of osteoclast precursor cells by the sequential expression of c-Fms and receptor activator of nuclear factor kappaB (RANK) receptors. 1999. J. Exp. Med. 190:1741-1754.
Jimi E., Aoki, K., Saito, H., D'Acquisto, F., May, M. J., Nakamura, I., Sudo, T., Kojima, T., Okamoto, F., Fukushima, H., Okabe, K., Ohya, K., and Ghosh, S. Selective inhibition of NF-kappa B blocks osteoclastogenesis and prevents inflammatory bone destruction in vivo. 2004. Nat. Med. 10:617-624.
Hoek, R. M., Ruuls, S. R., Murphy, C. A., Wright, G. J., Goddard, R., Zurawski, S. M., Blom, B., Homola, M. E., Streit, W. J., Brown, M. H., Barclay, A. N., Sedgwick, J. D. Down-regulation of the macrophage lineage through interaction with OX2 (CD200). 2000. Science 290:1768-1771.
Yagi, M., Miyamoto, T., Sawatani, Y., Iwamoto, K., Hosogane, N., Fujita, N., Morita, K., Ninomiya, K., Suzuki, T., Miyamoto, K., Oike, Y., Takeya, M., Toyama, Y., and Suda, T. DC-STAMP is essential for cell-cell fusion in osteoclasts and foreign body giant cells. 2005. J. Exp. Med. 202:345-351.
Chung, Y. H., Means, R. E., Choi, J. K., Lee, B. S., and Jung, J. U. Kaposi's sarcoma-associated herpesvirus OX2 glycoprotein activates myeloid-lineage cells to induce inflammatory cytokine production. 2002 J Virol 76:4688-4698
Foster-Cuevas, M., Wright, G. J., Puklavec, M. J., Brown, M. H., and Barclay, A. N. Human herpesvirus 8 K14 protein mimics CD200 in down-regulating macrophage activation through CD200 receptor. 2004. J. Virol. 78:7667-7676.
Cameron, C. M., Barrett, J. W., Liu, L., Lucas, A. R., and McFadden, G. Myxoma virus M141R expresses a viral CD200 (vOX-2) that is responsible for down-regulation of macrophage and T-cell activation in vivo. 2005. J. Virol. 79:6052-6067.
Shiratori, I. Yamaguchi, M., Suzukawa, M., Yamamoto, K., Lanier, L. L., Saito, T., and Arase, H. Down-regulation of basophil function by human CD200 and human herpesvirus-8 CD200. 2005. J. Immunol. 175:4441-4449.
Li, H., Cuartas, C., Cui, W., Choi, Y., Crawford, D. T., Ke, H.-Z., Kobayashi, K. S., Flavell, R. A., and Vignery, A. Interleukin-1-associated kinase M is a central regulator of osteoclast differentiation and activation. 2005. J. Exp. Med. 201:1169-1177.
Ballica, R., Valentijn, K., Khachatryan, A., Guerder, S., Kapadia, S., Gundberg, C., Gilligan, J., Flavell, R. A., and Vignery, A. Targeted expression of calcitonin gene related peptide to osteoblasts increases bone density in mice. 1999. J. Bone Mineral Res. 14: 1067-1074.
Baron, R., Vignery, A., Neff, L., Silvergate, A., and Santa Maria, A. Processing of undecalcified bone specimens for bone histomorphometry. 1982 In Bone Histomorphometry: Techniques and Interpretation. R R Recker, H M Frost, Eds. CRC Press, Inc., Boca Raton, Fla. 13-35.
Morinobu M, Nakamoto T, Hino K, Tsuji K, Shen Z J, Nakashima K, Nifuji A, Yamamoto H, Hirai H, and Noda M. The nucleocytoplasmic shuttling protein CIZ reduces adult bone mass by inhibiting bone morphogenetic protein-induced bone formation. 2005. J. Exp. Med. 201:961-970.
Voehringer, D., Rosen, D. B., Lanier, L. L., and Locksley, R. M. CD200 receptor family members represent novel DAP12-associated activating receptors on basophils and mast cells. 2004. J. Biol. Chem. 279:54117-54123.
Pear, W. S., Miller, J. P., Xu, L., Pui, J. C., Soffer, B., Quackenbush, R. C., Pendergast, A. M., Bronson, R., Aster, J. C., Scott, M. L., and Baltimore, D. Efficient and rapid induction of a chronic myelogenous leukemia-like myeloproliferative disease in mice receiving P210 bcr/abl-transduced bone marrow. 1998. Blood. 92:3780-3792.
Zar, J. H. 1984. Biostatistical Analysis, Englewood Cliffs, Prentice-Hall, N.J.
van den Berg, T. K., van Beek, E. M., Buhring, H. J., Colonna, M., Hamaguchi, M., Howard, C. J., Kasuga, M., Liu, Y., Matozaki, T., Neel, B. G., Parkos, C. A., Sano, S., Vignery, A., Vivier, E., Wright, M., Zawatzky, R., Barclay, A. N. A nomenclature for signal regulatory protein family members. 2005. J. Immunol. 175:7788-7789.
Lee, L., Liu, J., Manuel, J., and Gorczynski, R. M. A role for the immunomodulatory molecules CD200 and CD200R in regulating bone formation. 2006. Immunology letters 105:150-158.
Rubinstein, E., Ziyyat, A., Wolf, J. P., Le Naour, F., and Boucheix, C. The molecular players of sperm-egg fusion in mammals. 2006. Semin. Cell Dev. Biol. 17:254-63. Epub 2006 Mar. 2. Review.
Inoue, N., Ikawa, M., Isotani, A., and Okabe, M. immunoglobulin superfamily protein Izumo is required for sperm to fuse with eggs. 2005. Nature. 434:234-238.
Hatherley, D., Cherwinski, H. M., Moshref, M., Barclay, A. N. Recombinant CD200 protein does not bind activating proteins closely related to CD200 receptor. 2005. J. Immunol. 175:2469-2474.
Hatherley, D., Barclay, A. N. CD200 and CD200 receptor cell surface proteins interact through their N-terminal immunoglobulin-like domains. 2004. Eur. J. Immunol. 34:1688-1694.
Wright, G. J., Puklavec, M. J., Willis, A. C., Hoek, R. M., Sedgwick, J. D., Brown, M. H., and Barclay, A. N. Lymphoid/neuronal cell surface OX2 glycoprotein recognizes a novel receptor on macrophages implicated in the control of their function. 2000. Immunity. 13:233-242.
Wright, G. J., Cherwinski, H., Foster-Cuevas, M., Brooke, G., Puklavec, M. J., Bigler, M., Song, Y., Jenmalm, M., Gorman, D., McClanahan, T., Liu, M. R., Brown, M. H., Sedgwick, J. D., Phillips, J. H., Barclay, A. N. Characterization of the CD200 receptor family in mice and humans and their interactions with CD200. 2003. J. Immunol. 171:3034-3046.
Parkinson, J. E., Sanderson, C. M., and Smith G. L. The vaccinia virus A38L gene product is a 33 kD integral membrane glycoprotein. 1995. Virology. 214: 177-188.
Cameron, C. M., Barrett, J. W., Mann, M., Lucas, A., and McFadden, G. Myxoma virus M128L is expressed as a cell surface CD47-like virulence factor that contributes to the downregulation of macrophage activation in vivo. 2005. Virology. 337:55-67.

While osteoclasts and giant cells have long been recognized, the molecular mechanism by which their mononucleated precursors adhere and fuse with each other, a key step in their differentiation, remains poorly understood. Indeed, cell-cell fusion itself, whether it concerns that of sperm-oocyte or myoblast-myoblast, leading to fertilization and muscle development respectively, has not been thoroughly investigated. It is thought that cell-cell adhesion leading to fusion involves a set of proteins similar to those used by viruses to fuse with host cells and inject their DNA or RNA (Hernandez et al, 1997). It has been hypothesized that viruses have stolen the fusion protein machinery from their target cells. It is now well accepted that virus-cell fusion requires both an attachment mechanism and a fusion peptide. One such example is HIV gp120 from the human immunodeficiency virus which binds CD4 on T lymphocytes and macrophages (Dalgleish et al., 1984; Klatzmann et al., 1984) while the fusion molecule gp40, which arises from the same precursor molecule (gp160) is thought to trigger the actual fusion event. While putative fusion molecules mediating sperm-oocyte and myoblast fusion have been reported (Blobel et al., 1992; Wakelam 1989), the actual protein machinery governing the attachment and fusion of these cells remains unknown.

Of relevance to the fusion of macrophages, the 100 kD form of CD44, the most common so-called “standard form” expressed by hematopoietic cells, is involved not only in the attachment of poliovirus to HeLa cells (Shepley and Racaniello, 1994) but also in the infection of mononuclear phagocytes by HIV (Rivandeneira et al, 1995). CD44 does not, however, act as a viral receptor in either of these two instances.

MFR is a type I transmembrane glycoprotein that belongs to the superfamily of immunoglobulins (Ig) (Saginario et al., 1998). MFR contains three Ig domains in its extracellular part, and closely resembles CD4.

CD47, the ligand for MFR/SIRPαs proteins expressed vy Vaccinia and Variola viruses (Parkinson et al., 1995). Although A38L is not known as the actual fusion protein, like CD47, A38L promotes Ca⁺⁺ entry into cells possibly by forming a pore (Sanderson et al., 1996). Indeed, pore formation is a classical tactic used by parasites to enter host cells (Kirby et al., 1998). Of note, the overexpression of the pore forming P2Z/P2X₇ receptor for ATP leads to cell-cell fusion, but is followed by cell death. Likewise, the overexpression of CD47 or A38L leads to cell death (Nishiyama et al., 1997). This raises the possibility that once the membranes from opposite cells are closely apposed and stable, CD47 molecules may create a pore that triggers cell-cell fusion. While this last possibility is highly speculative, it opens an interesting avenue of research.

CD200-like genes have been identified for some, but not all, members of the double-stranded DNA virus families of poxviruses, herpesviruses, and adenoviruses (Chung et al. 2002; Foster-Cuevas et al. 2004). It is now known that Kaposi's sarcome-associated herpesvirus (KSHV/HHV-8)-K14 gene product is a ligand for CD200R (chung, 2002; foster-cuevas, 2004). Similarly, M141R is a myxoma virus gene that encodes a cell surface protein with significant amino acid similarity to the CD200, and is required for the full pathogenesis of myxoma virus in the European rabbit Camron et al. 2005).

### Methods of Use

As mentioned in the introduction section, osteoclasts are essential for both the development and the remodeling of bone, and increases in the number and/or activity of osteoclasts lead to diseases that are associated with generalized bone loss. The invention therefore provides for a method of treating a patient with a disease associated with generalized bone loss, such as osteoporosis, and others that are associated with localized bone loss, such as rheumatoid arthritis and periodontal disease.

The invention therefore provides for a method of treating a patient with cancer with bone metastases. Breast and prostate cancers are the leading causes of cancer death among women and men second only to lung cancer. Early detection and treatment of these cancers has increased the 5-year survival rate to 98% for breast cancer and 100% for prostate cancer when detected at the earliest stages. However, the breast cancer survival drops to 26% for patients initially diagnosed with distant metastases, while prostate cancer survival rate drops to 33% with distant metastases. The skeleton is a preferred site for breast and prostate cancer metastasis. Many other common cancers, including lung and renal tumors, melanoma, and multiple myeloma also attack the skeleton.

There are four major targets for therapeutic intervention against bone metastases: (A) the tumor cells themselves, but also (B) osteoclastic bone resorption; (C) the activity of osteoblasts; and (D) the specific bone microenvironment surrounding the tumor cells themselves. Targeting osteoclasts forms the basis for approved clinical treatments of all tumor types that attack the skeleton. Current clinical treatments for established bone metastases are palliative. They effectively reduce metastases and improve patient quality of life, but they do not increase survival. We now also appreciate that most types of cancer treatment cause bone loss, and that a major morbidity in patients with bone metastases is intractable pain

Regulating fusion of macrophages to prevent the formation of cancer-associated bone metastases is essential.

### REFERENCES

John M. Chirgwin and Theresa A. Guise. Skeletal Metastases: Decreasing Tumor Burden by Targeting the Bone Microenvironment. Journal of Cellular Biochemistry 102:1333-1342 (2007)

### Foreign Body Giant Cell

Multinucleated giant cells have long been regarded as hallmark histological features of chronic inflammation arising from the persistent presence of foreign microorganisms, materials, pathogens or otherwise undefined etiological agents. They are formed from blood monocyte-derived macrophage macrophage fusion in the chronic inflammatory setting by a mechanism that is as yet unclear and for physiological reasons that are also uncertain. However, foreign body giant cell formation on implanted biomaterials is associated with material degradation and biomedical device failure and is therefore an undesirable consequence of the chronic inflammatory response to biomedical polymers.

Regulating fusion of macrophages to prevent the deleterious effects of giant cells on implanted biomaterials and devices, whether sensing or delivering molecules, is essential.

### REFERENCES

Amy K. McNally, James M. Anderson. Multinucleated giant cell formation exhibits features of phagocytosis with participation of the endoplasmic reticulum. Experimental and Molecular Pathology 79:126-135 (2005)

The invention therefore provides for a method of treating a patient with giant cell tumor. Giant cell tumor (GCT) of bone, also known as osteoclastoma, is a primary osteolytic bone neoplasm in which monocytic macrophage/osteoclast precursor cells and multinucleated osteoclast-like giant cells infiltrate the tumor. GCT also occur in non osseous tissues, such as in the uterus. The origin of GCT is unknown, but the tumor cells of GCT have been reported to produce chemoattractants that can attract osteoclasts and their precursors. It has been speculated that GCT originate from the fusion of cells that belong to the monocyte/macrophage lineage with themselves and with tumor cells.

GCT is usually benign but locally aggressive, and most commonly occurs in the epiphysis of long bones. Rarely, GCT can originate at extra osseous sites. Metastases from GCT of bone are unusual, and often behave in an indolent manner that can be managed by surgery. More rarely, GCT may exhibit a much more aggressive phenotype.

Regulating fusion of macrophages to prevent the formation of giant cell tumors is essential.

### REFERENCES

Skubitz K M, Manivel J C. Giant cell tumor of the uterus: case report and response to chemotherapy. BMC Cancer 7:46. Review. (2007)

A composition according to the invention, may comprise a CD200/CD200R, agonist, an antagonist, a CD200-based biotherapeutic, an activating antibody or fragment that promotes the activation of the pathway. For therapeutic use, the compositions may be administered in any conventional dosage form in any conventional manner. Routes of administration include, but are not limited to, intravenously, intramuscularly, subcutaneously, intrasynovially, by infusion, sublingually, transdermally, orally, topically or by inhalation. The preferred modes of administration are oral and intravenous. The compositions may be administered alone or in combination with adjuvants that enhance stability of the inhibitors, facilitate administration of pharmaceutic compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies. The above described compositions may be physically combined with the conventional therapeutics or other adjuvants into a single pharmaceutical composition. Advantageously, the compositions may then be administered together in a single dosage form. In some embodiments, the pharmaceutical compositions comprising such combinations of compositions contain at least about 5%, but more preferably at least about 20%, of a composition (w/w) or a combination thereof. The optimum percentage (w/w) of a composition of the invention may vary and is within the purview of those skilled in the art. Alternatively, the compositions may be administered separately (either serially or in parallel). Separate dosing allows for greater flexibility in the dosing regime.

As mentioned above, dosage forms of the compositions described herein include pharmaceutically acceptable carriers and adjuvants known to those of ordinary skill in the art. These carriers and adjuvants include, for example, ion exchangers, alumina, aluminum stearate, lecithin, serum proteins, buffer substances, water, salts or electrolytes and cellulose-based substances. Preferred dosage forms include, tablet, capsule, caplet, liquid, solution, suspension, emulsion, lozenges, syrup, reconstitutable powder, granule, suppository and transdermal patch. Methods for preparing such dosage forms are known (see, for example, H. C. Ansel and N. G. Popovish, Pharmaceutical Dosage Forms and Drug Delivery Systems, 5th ed., Lea and Febiger (1990)). Dosage levels and requirements are well-recognized in the art and may be selected by those of ordinary skill in the art from available methods and techniques suitable for a particular patient. In some embodiments, dosage levels range from about 1-1000 mg/dose for a 70 kg patient. Although one dose per day may be sufficient, up to 5 doses per day may be given. For oral doses, up to 2000 mg/day may be required. As the skilled artisan will appreciate, lower or higher doses may be required depending on particular factors. For instance, specific dosage and treatment regimens will depend on factors such as the patient's general health profile, the severity and course of the patient's disorder or disposition thereto, and the judgment of the treating physician.

1. A biotherapeutic composition comprising CD200 protein or it's receptor protein or fragments thereof, wherein the biotherapeutic composition activates or inhibits the CD200 pathway.

2. An antibody or antibody binding site which effectively binds CD200 protein or it's receptor or fragments thereof, wherein the above antibody or antibody binding site which binds CD200 or its receptor inhibits differentiation of osteoclasts.

3. An antibody or antibody binding site which effectively binds CD200 protein or it's receptor protein or fragments thereof, wherein the above antibody or antibody binding site which binds CD200 or its receptor activates the CD200 pathway.

4. A method of treating disease or condition chosen from osteoporosis, Paget's disease, metastatic cancers wherein the skeleton is a preferred site for metastasis, diseases or conditions wherein multinucleated giant cells have a negative effect and giant cell tumor the method comprising administering to a patient a therapeutically effective amount of a biotherapeutic composition according to claim 1 which is an agonist of CD200 and its receptor interaction or an antibody according to claim 3.

5. The method according to claim 4 wherein the disease or condition chosen from osteoporosis, Paget's disease, breast cancer, prostate cancer, lung tumors, renal tumors, melanoma, multiple myeloma and chronic inflammatory response to implantations.

6. A method of treating diseases associated with generalized bone loss, the method comprising administering to a patient a therapeutically effective amount of a biotherapeutic composition according to claim 1 which inhibits CD200 and its receptor interaction or an antibody according to claim 2.

7. The method according to claim 6 wherein the diseases are chosen from osteoporosis, rheumatoid arthritis and periodontal disease.

8. A method to identify a compound that inhibits interaction of CD200 and its receptor in a cell, comprising: (1) contacting a cell with a putative regulatory compound, wherein the cell includes a CD200 and its receptor protein; and (2) assessing the ability of the putative regulatory compound to inhibit the interaction of CD200 and its receptor.

9. A method to identify a compound that is an agonist of CD200 interaction with its receptor in a cell, comprising: (1) contacting a cell with a putative regulatory compound, wherein the cell includes a CD200 and its receptor protein; and (2) assessing the ability of the putative regulatory compound to activate the CD200 pathway.
